(19) United States
(12) Patent Application Publication
(10) Pub. No.: US 2026/0335642 A1
(43) Pub. Date: Oct. 29, 2026

(54) METHOD FOR PROVIDING TRANSCUTANEOUS AURICULAR VAGUS NERVE STIMULATION TO EPILEPSY PATIENT AND CONTROL APPARATUS

(71) Applicants: SAMSUNG LIFE PUBLIC WELFARE FOUNDATION, Seoul (KR); SOONCHUNHYANG UNIVERSITY INDUSTRY ACADEMY COOPERATION FOUNDATION, Asan-si (KR)

(72) Inventors: Sung Jun HONG, Seoul (KR); Young Min SHON, Seoul (KR); Jung Hun KIM, Seoul (KR); Tae Kyung KIM, Seoul (KR); Do Kyoon KIM, Seoul (KR); Jung Seok CHOI, Seoul (KR); Eunkyoung PARK, Asan-si (KR); Eunmi CHOI, Asan-si (KR)

(21) Appl. No.: 19/654,695

(22) Filed: Apr. 22, 2026

(30) Foreign Application Priority Data
Apr. 23, 2025 (KR) 10-2025-0053007

(51) Int. Cl. A61N 1/36

(52) U.S. Cl. CPC A61N 1/36025; A61N 1/36031; A61N 1/36034

(57) ABSTRACT

A method of controlling vagus nerve electrical stimulation for an epilepsy patient includes: receiving, by a control apparatus, electrocardiogram (ECG) data of an epilepsy patient at a specific point in time; extracting, by the control apparatus, a heart rate variability (HRV) parameter from the ECG data; computing, by the control apparatus, a change amount between an HRV parameter in a normal state of the epilepsy patient and an HRV parameter at the specific point in time; and determining, by the control apparatus, stimulation parameter information for vagus nerve electrical stimulation of the epilepsy patient based on the change amount.

### CROSS REFERENCE TO RELATED APPLICATIONS

This application claims priority to Korean Patent Application No. 10-2025-0053007, filed on Apr. 23, 2025, the entire contents of which are hereby incorporated by reference.

### BACKGROUND

### 1. Field

The following description relates to a transcutaneous auricular vagus nerve stimulation (taVNS) technique for providing dynamic stimulation intensity according to the condition of an epilepsy patient. The following description also relates to a technique for controlling taVNS intensity for an epilepsy patient.

### 2. Description of the Related Art

Epilepsy is a brain disease in which symptoms such as seizures caused by temporary abnormal excitation of brain neurons occur chronically and repeatedly. Epilepsy is primarily treated with medication. However, for seizures in patients with refractory epilepsy who do not respond to medication, transcutaneous auricular vagus nerve stimulation (taVNS) therapy may be used.

### SUMMARY

In one general aspect, there is provided a method of providing vagus nerve electrical stimulation to an epilepsy patient includes: receiving, by a transcutaneous auricular vagus nerve stimulator, electrocardiogram (ECG) data of an epilepsy patient at a specific point in time; extracting, by the transcutaneous auricular vagus nerve stimulator, a heart rate variability (HRV) parameter from the ECG data; computing, by the transcutaneous auricular vagus nerve stimulator, a change amount between an HRV parameter in a normal state of the epilepsy patient and an HRV parameter at the specific point in time; generating, by the transcutaneous auricular vagus nerve stimulator, stimulation parameter information for vagus nerve electrical stimulation of the epilepsy patient based on the change amount; and providing, by the transcutaneous auricular vagus nerve stimulator, vagus nerve electrical stimulation to the epilepsy patient according to the stimulation parameter information.

In another aspect, there is provided a method of controlling vagus nerve electrical stimulation for an epilepsy patient includes: receiving, by a control apparatus, ECG data of an epilepsy patient at a specific point in time; extracting, by the control apparatus, an HRV parameter from the ECG data; computing, by the control apparatus, a change amount between an HRV parameter in a normal state of the epilepsy patient and an HRV parameter at the specific point in time; and determining, by the control apparatus, stimulation parameter information for vagus nerve electrical stimulation of the epilepsy patient based on the change amount, wherein the stimulation parameter information for the vagus nerve electrical stimulation comprises at least one of stimulation intensity, frequency, pulse width, and stimulation duration.

In yet another aspect, there is provided a control apparatus for vagus nerve electrical stimulation for an epilepsy patient includes: an interface device configured to receive ECG data of an epilepsy patient at a specific point in time; a storage device configured to store ECG data of the epilepsy patient in a normal state; and a processor configured to compute a change amount between an HRV parameter in the normal state derived from the ECG data in the normal state of the epilepsy patient and an HRV parameter at the specific point in time derived from the received ECG data, and to compute stimulation parameter information for vagus nerve electrical stimulation of the epilepsy patient based on the change amount.

### BRIEF DESCRIPTION OF THE DRAWINGS

FIG. 1 illustrates an example of a taVNS system for an epilepsy patient.

FIG. 2 illustrates an example of a taVNS device for an epilepsy patient.

FIG. 3 illustrates an example of a process of extracting HRV parameters from ECG data.

FIG. 4 illustrates an example of intervals in which ECG data are collected.

FIGS. 5A and 5B show results of analyzing time-domain HRV variability in a taVNS group.

FIGS. 6A and 6B show results of analyzing frequency-domain HRV variability in a taVNS group.

FIGS. 7A and 7B show immunohistochemistry results and analysis of variance results for a normal group, a PTZ group, and a taVNS group.

FIG. 8 illustrates an example of a control apparatus for controlling taVNS conditions.

Throughout the drawings and the detailed description, unless otherwise described, the same drawing reference numerals should be understood to refer to the same elements, features, and structures. The sizes of regions and elements, and depiction thereof may be exaggerated for clarity, illustration, and/or convenience.

### DETAILED DESCRIPTION

The following detailed description is provided to assist the reader in gaining a comprehensive understanding of the methods, apparatuses, and/or systems described herein. Accordingly, various changes, modifications, and equivalents of the systems, apparatuses and/or methods described herein will be understood by those of ordinary skill in the art.

Moreover, descriptions of well-known functions and constructions may be omitted for increased clarity and conciseness. Further, repetitive descriptions may be omitted for brevity. The progression of processing steps and/or operations described is a non-limiting example.

The sequence of steps and/or operations is not limited to that set forth herein and may be changed to occur in an order that is different from an order described herein, with the exception of steps and/or operations necessarily occurring in a particular order. In one or more examples, two operations in succession may be performed substantially concurrently, or the two operations may be performed in a reverse order or in a different order depending on a function or operation involved.

Unless stated otherwise, like reference numerals may refer to like elements throughout even when they are shown in different drawings. Unless stated otherwise, the same reference numerals may be used to refer to the same or substantially the same elements throughout the specification and the drawings. In one or more aspects, identical elements (or elements with identical names) in different drawings may have the same or substantially the same functions and properties unless stated otherwise. Names of the respective elements used in the following explanations are selected only for convenience and may be thus different from those used in actual products.

Advantages and features of the present disclosure, and implementation methods thereof, are clarified through the embodiments described with reference to the accompanying drawings. The present disclosure may, however, be embodied in different forms and should not be construed as limited to the embodiments set forth herein. Rather, these embodiments are examples and are provided so that this disclosure may be thorough and complete to assist those skilled in the art to understand the inventive concepts without limiting the protected scope of the present disclosure.

Shapes, dimensions (e.g., sizes, lengths, locations, and areas), proportions, ratios, numbers, the number of elements, and the like disclosed herein, including those illustrated in the drawings, are merely examples, and thus, the present disclosure is not limited to the illustrated details. It is, however, noted that the relative dimensions of the components illustrated in the drawings are part of the present disclosure.

When the term “comprise,” “have,” “include,” “contain,” “constitute,” “made of,” “formed of,” “composed of,” or the like is used with respect to one or more elements (e.g., components, structures, groups, circuits, networks, members, parts, areas, portions, integers, steps, operations, and/or the like), one or more other elements may be added unless a term such as “only” or the like is used. The terms used in the present disclosure are merely used in order to describe particular example embodiments, and are not intended to limit the scope of the present disclosure. The terms of a singular form may include plural forms unless the context clearly indicates otherwise. For example, an element may be one or more elements. An element may include a plurality of elements. The word “exemplary” is used to mean serving as an example or illustration. Embodiments are example embodiments. Aspects are example aspects. In one or more implementations, “embodiments,” “examples,” “aspects,” and the like should not be construed to be preferred or advantageous over other implementations. An embodiment, an example, an example embodiment, an aspect, or the like may refer to one or more embodiments, one or more examples, one or more example embodiments, one or more aspects, or the like, unless stated otherwise. Further, the term “may” encompasses all the meanings of the term “can.”

In one or more aspects, unless explicitly stated otherwise, an element, feature, or corresponding information (e.g., a level, range, dimension, or the like) is construed to include an error or tolerance range even where no explicit description of such an error or tolerance range is provided. An error or tolerance range may be caused by various factors (e.g., process factors, internal or external impact, noise, or the like). In interpreting a numerical value, the value is interpreted as including an error range unless explicitly stated otherwise.

When a positional relationship between two elements (e.g., components, structures, groups, circuits, networks, members, parts, areas, portions, and/or the like) are described using any of the terms such as “adjacent to,” “beside,” “next to,” and/or the like indicating a position or location, one or more other elements may be located between the two elements unless a more limiting term, such as “immediate(ly),” “direct(ly),” or “close(ly),” is used. Furthermore, the spatially relative terms such as the foregoing terms as well as other terms such as “column,” “row,” “vertical,” “horizontal,” “diagonal,” and the like refer to an arbitrary frame of reference.

In describing a temporal relationship, when the temporal order is described as, for example, “after,” “following,” “subsequent,” “next,” “before,” “preceding,” “prior to,” or the like, a case that is not consecutive or not sequential may be included and thus one or more other events may occur therebetween, unless a more limiting term, such as “just,” “immediate(ly),” or “direct(ly),” is used.

It is understood that, although the terms “first,” “second,” and the like may be used herein to describe various elements (e.g., components, structures, groups, circuits, networks, members, parts, areas, portions, and/or the like), these elements should not be limited by these terms, for example, to any particular order, precedence, or number of elements. These terms are used only to distinguish one element from another. For example, a first element may denote a second element, and, similarly, a second element may denote a first element, without departing from the scope of the present disclosure. Furthermore, the first element, the second element, and the like may be arbitrarily named according to the convenience of those skilled in the art without departing from the scope of the present disclosure. For clarity, the functions or structures of these elements (e.g., the first element, the second element, and the like) are not limited by ordinal numbers or the names in front of the elements. Further, a first element may include one or more first elements. Similarly, a second element or the like may include one or more second elements or the like.

In describing elements of the present disclosure, the terms “first,” “second,” “A,” “B,” “(a),” “(b),” or the like may be used. These terms are intended to identify the corresponding element(s) from the other element(s), and these are not used to define the essence, basis, order, or number of the elements.

The expression that an element (e.g., component, structure, group, circuit, network, member, part, area, portion, and/or the like) “is engaged” with another element may be understood, for example, as that the element may be either directly or indirectly engaged with the another element. The term “is engaged” or similar expressions may refer to a term such as “is connected,” “is coupled,” “is combined,” “is linked,” “is provided,” “interacts,” or the like. The engagement may involve one or more intervening elements disposed or interposed between the element and the another element, unless otherwise specified.

The terms such as a “line” or “direction” should not be interpreted only based on a geometrical relationship in which the respective lines or directions are parallel, perpendicular, diagonal, or slanted with respect to each other, and may be meant as lines or directions having wider directivities within the range within which the components of the present disclosure may operate functionally.

The term “at least one” should be understood as including any and all combinations of one or more of the associated listed items. For example, each of the phrases “at least one of a first item, a second item, or a third item” and “at least one of a first item, a second item, and a third item” may represent (i) a combination of items provided by two or more of the first item, the second item, and the third item or (ii) only one of the first item, the second item, or the third item. Further, at least one of a plurality of elements can represent (i) one element of the plurality of elements, (ii) some elements of the plurality of elements, or (iii) all elements of the plurality of elements. Further, “at least some,” “at least some portions,” “at least some parts,” “at least a portion,” “at least one or more portions,” “at least a part,” “at least one or more parts,” “at least some elements,” “one or more,” or the like of a plurality of elements can represent (i) one element of the plurality of elements, (ii) a portion (or a part) of the plurality of elements, (iii) one or more portions (or parts) of the plurality of elements, (iv) multiple elements of the plurality of elements, or (v) all of the plurality of elements. Moreover, “at least some,” “at least some portions,” “at least some parts,” “at least a portion,” “at least one or more portions,” “at least a part,” “at least one or more parts,” or the like of an element can represent (i) a portion (or a part) of the element, (ii) one or more portions (or parts) of the element, or (iii) the element, or all portions of the element.

The expression of a first element, a second elements “and/or” a third element should be understood as one of the first, second and third elements or as any or all combinations of the first, second and third elements. By way of example, A, B and/or C may refer to only A; only B; only C; any of A, B, and C (e.g., A, B, or C); some combination of A, B, and C (e.g., A and B; A and C; or B and C); or all of A, B, and C. Furthermore, an expression “A/B” may be understood as A and/or B. For example, an expression “A/B” may refer to only A; only B; A or B; or A and B.

In one or more aspects, the terms “between” and “among” may be used interchangeably simply for convenience unless stated otherwise. For example, an expression “between a plurality of elements” may be understood as among a plurality of elements. In another example, an expression “among a plurality of elements” may be understood as between a plurality of elements. In one or more examples, the number of elements may be two. In one or more examples, the number of elements may be more than two. Furthermore, when an element is referred to as being “between” at least two elements, the element may be the only element between the at least two elements, or one or more intervening elements may also be present.

In one or more aspects, the phrases “each other” and “one another” may be used interchangeably simply for convenience unless stated otherwise. For example, an expression “different from each other” may be understood as being different from one another. In another example, an expression “different from one another” may be understood as being different from each other. In one or more examples, the number of elements involved in the foregoing expression may be two. In one or more examples, the number of elements involved in the foregoing expression may be more than two.

In one or more aspects, the phrases “one or more among” and “one or more of” may be used interchangeably simply for convenience unless stated otherwise.

The term “or” means “inclusive or” rather than “exclusive or.” That is, unless otherwise stated or clear from the context, the expression that “x uses a or b” means any one of natural inclusive permutations. For example, “a or b” may mean “a,” “b,” or “a and b.” For example, “a, b or c” may mean “a,” “b,” “c,” “a and b,” “b and c,” “a and c,” or “a, b and c.”

A phrase “substantially the same” may indicate a degree of being considered as being equivalent to each other taking into account minute differences due to errors in the manufacturing or operating process.

Features of various embodiments of the present disclosure may be partially or entirely coupled to or combined with each other, may be technically associated with each other, and may be variously operated, linked or driven together in various ways. Embodiments of the present disclosure may be implemented or carried out independently of each other or may be implemented or carried out together in a co-dependent or related relationship. In one or more aspects, the components of each apparatus and device according to various embodiments of the present disclosure are operatively coupled and configured.

The terms used herein have been selected as being general in the related technical field; however, there may be other terms depending on the development and/or change of technology, convention, preference of technicians, and so on. Therefore, the terms used herein should not be understood as limiting technical ideas, but should be understood as examples of the terms for describing example embodiments.

Further, in a specific case, a term may be arbitrarily selected by an applicant, and in this case, the detailed meaning thereof is described herein. Therefore, the terms used herein should be understood based on not only the name of the terms, but also the meaning of the terms and the content hereof.

In the following description, various example embodiments of the present disclosure are described in more detail with reference to the accompanying drawings. With respect to reference numerals to elements of each of the drawings, the same elements may be illustrated in other drawings, and like reference numerals may refer to like elements unless stated otherwise. The same or similar elements may be denoted by the same reference numerals even though they are depicted in different drawings. In addition, for the convenience of description, a scale and dimension of each of the elements illustrated in the accompanying drawings may be different from an actual scale and dimension, and thus, embodiments of the present disclosure are not limited to a scale and dimension illustrated in the drawings.

Before starting detailed explanations of figures, components that will be described in the specification are distinguished merely according to functions mainly performed by the components. That is, two or more components which will be described later can be integrated into a single component. Furthermore, a single component which will be explained later can be separated into two or more components. Moreover, each component which will be described can additionally perform some or all of a function executed by another component in addition to the main function thereof. Some or all of the main function of each component which will be explained can be carried out by another component. Accordingly, presence/absence of each component which will be described throughout the specification should be functionally interpreted.

The following description relates to a system for controlling vagus nerve electrical stimulation in a closed-loop manner based on an electrocardiogram signal measured in real time.

The following description relates to a taVNS device control technique.

The following description relates to a taVNS intensity control technique for epilepsy patients.

In the following description, a control apparatus determines patient-customized taVNS intensity. The control apparatus may be implemented in various forms of devices. For example, the control apparatus may be implemented in the form of a PC, a smart device, a wearable device, a server, a cloud server, a data processing dedicated chipset, or the like.

The control apparatus may form a closed-loop control structure that continuously receives ECG data and repeatedly updates stimulation conditions by comparing the HRV parameter at a previous point in time with the current HRV parameter. Accordingly, the control apparatus may perform adaptive control that automatically adjusts stimulation conditions in response to changes in the patient's condition without external input.

Vagus nerve stimulation (VNS) is widely known as a neuromodulation technology for treating various neurological diseases through autonomic nervous system regulation.

Transcutaneous auricular vagus nerve stimulation (taVNS) is a technology that activates vagal signal transduction pathways by non-invasively stimulating the auricular branch of the vagus nerve distributed in the auricle region of the ear.

The vagal signal stimulated by taVNS is transmitted to the nucleus tractus solitarius (NTS) of the medulla oblongata, and then diffuses to various brain regions including the locus coeruleus, hypothalamus, and hippocampus, thereby regulating the balance between the sympathetic and parasympathetic systems of the autonomic nervous system. Through such a neurological pathway, taVNS may alter heart rate variability (HRV) and exhibit an effect of regulating autonomic nervous system function.

HRV serves as an indicator of autonomic nervous system balance, reflecting the interaction between sympathetic and parasympathetic activities.

The taVNS parameter information or stimulation parameter information may include at least one of stimulation intensity, frequency, pulse width, and stimulation duration.

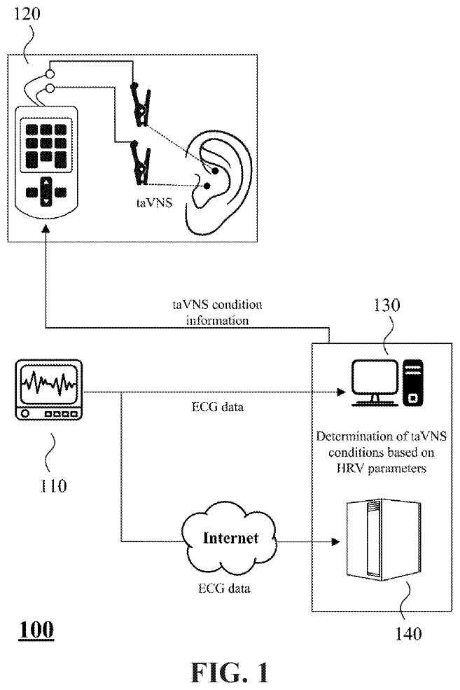

FIG. 1 illustrates an example of a taVNS system 100 for an epilepsy patient. In FIG. 1, the control apparatus takes the form of a computer terminal 130 such as a PC and a server 140.

An electrocardiogram device 110 acquires electrocardiogram (ECG) data of an epilepsy patient. The electrocardiogram device 110 may continuously monitor the electrocardiogram of the epilepsy patient.

A taVNS device 120 provides vagus nerve electrical stimulation to the epilepsy patient.

The computer terminal 130 may receive the ECG data of the epilepsy patient from the electrocardiogram device 110. The computer terminal 130 may determine taVNS conditions (stimulation intensity, frequency, duration, etc.) based on the ECG data of the epilepsy patient. The computer terminal 130 may extract HRV parameters from the ECG data of the epilepsy patient.

The computer terminal 130 may determine taVNS conditions (stimulation intensity, frequency, pulse width, duration, etc.) based on HRV parameters. The computer terminal 130 may determine taVNS conditions (stimulation intensity, frequency, pulse width, duration, etc.) based on HRV parameters over a predetermined time interval. Accordingly, the computer terminal 130 may determine taVNS conditions (stimulation intensity, frequency, duration, etc.) over a predetermined time flow. At this time, the taVNS intensity and the like may be dynamically changed based on the HRV parameters of the patient. The computer terminal 130 may transmit the taVNS stimulation parameter information determined at a certain point in time to the taVNS device 120.

The computer terminal 130 may update the taVNS conditions using HRV parameters extracted from consecutive time intervals.

The server 140 may receive the ECG data of the epilepsy patient from the electrocardiogram device 110. The server 140 may determine taVNS conditions (stimulation intensity, frequency, duration, etc.) based on the ECG data of the epilepsy patient. The server 140 may extract HRV parameters from the ECG data of the epilepsy patient. The server 140 may determine taVNS conditions (stimulation intensity, frequency, duration, etc.) based on HRV parameters. The server 140 may determine taVNS conditions (stimulation intensity, frequency, duration, etc.) based on HRV parameters over a predetermined time interval. Accordingly, the server 140 may determine taVNS conditions (stimulation intensity, frequency, duration, etc.) over a predetermined time flow. At this time, the taVNS intensity and the like may be dynamically changed based on the HRV parameters of the patient. The server 140 may transmit the taVNS stimulation parameter information determined at a certain point in time to the taVNS device 120.

The server 140 may update the taVNS conditions using HRV parameters extracted from consecutive time intervals.

The taVNS device 120 may adjust the electrical stimulation intensity according to the received stimulation stimulation parameter information to provide vagus nerve stimulation.

The taVNS device 120 may include a stimulation output circuit that generates an actual electrical stimulation signal according to the stimulation stimulation parameter information and delivers it to the auricle region of the patient through electrodes.

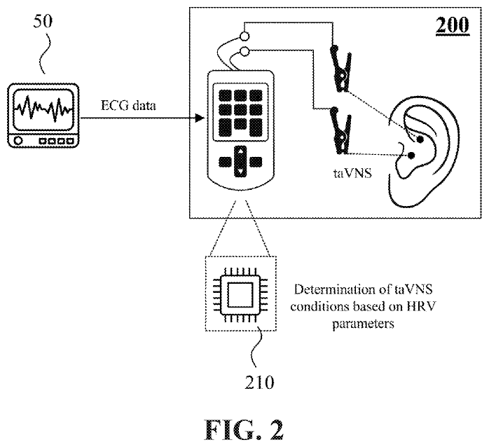

FIG. 2 illustrates an example of a taVNS device 200 for an epilepsy patient. The taVNS device 200 provides vagus nerve electrical stimulation to the epilepsy patient. In FIG. 2, the control apparatus takes the form of a microcontroller unit (MCU) 210, which is a chipset embedded in the taVNS device 200 or a chipset in which a control program is embedded.

The taVNS device 200 includes a microcontroller unit (MCU) 210. The taVNS device 200 may further include an electrical stimulation generation component, an electrical stimulation delivery component, a power supply component, and the like. The taVNS device 200 itself may include device components widely known in the relevant technical field.

An electrocardiogram device 50 acquires ECG data of the epilepsy patient. The electrocardiogram device 50 may continuously monitor the electrocardiogram of the epilepsy patient.

The MCU 210 may receive the ECG data of the epilepsy patient from the electrocardiogram device 50.

The MCU 210 may process data and determine taVNS intensity.

The MCU 210 may determine taVNS intensity based on the ECG data of the epilepsy patient.

The MCU 210 may extract HRV parameters from the ECG data of the epilepsy patient.

The MCU 210 may determine taVNS conditions (stimulation intensity, frequency, duration, etc.) based on HRV parameters.

The MCU 210 may determine taVNS conditions (stimulation intensity, frequency, duration, etc.) based on ECG data over a predetermined time interval. Accordingly, the MCU 210 may determine taVNS conditions over a predetermined time flow. At this time, the taVNS intensity and the like may be dynamically changed based on the ECG data of the patient.

The MCU 210 may update the taVNS conditions using HRV parameters extracted from consecutive time intervals.

The taVNS device 200 may adjust the taVNS conditions based on the taVNS stimulation parameter information determined by the MCU 210.

The control apparatus may determine taVNS conditions (stimulation intensity, frequency, duration, etc.) based on the heart rate variability (HRV) of the epilepsy patient. This presupposes that there is a certain correlation between the seizure state of an epilepsy patient and HRV. The following describes research results on the correlation between the seizure state of epilepsy patients and HRV.

This study used 16 male Sprague-Dawley rats aged 8 to 9 weeks and weighing 280 to 300 g. The experimental animal groups were divided into a normal group (n=5) anesthetized only with dexmedetomidine (DEX), a PTZ group (n=5) that received pentylenetetrazol (PTZ) injection without additional treatment, and a taVNS group (n=6) that received taVNS treatment after PTZ injection.

Electrocardiogram (ECG) signals were measured for 55 minutes for the subjects using a digital system (NicoletOne EEG System). The normal group collected baseline signals for 55 minutes after induction of anesthesia with DEX (0.3 mg/kg, intraperitoneal injection). The PTZ group collected baseline signals for 10 minutes after DEX administration, then induced epileptic seizures by injecting PTZ (45 mg/kg, intraperitoneal injection) and collected seizure characteristics and ECG changes for approximately 45 minutes. The taVNS group collected baseline signals for 10 minutes after DEX injection, collected seizure characteristic signals for 15 minutes after PTZ injection, and then performed taVNS (5-minute stimulation, 10-minute recovery) twice at 15-minute intervals while collecting signals.

Bipolar taVNS electrodes were fabricated using two stainless steel cylinder blocks with a diameter of 2 mm and a height of 5 mm, and biocompatible epoxy (EPOTEX 301). The electrodes were fabricated according to the guidelines of previous studies (J. Mun et al., Frequency-dependent depression of the NTS synapse affects the temporal response of the antihypertensive effect of auricular vagus nerve stimulation (aVNS), J. Neural Eng., vol. 19, no. 4, August 2022). Electrical stimulation was administered for 5 minutes at a frequency of 20 Hz, a pulse width of 0.5 ms, and an intensity of 2.5 mA using a stimulator (NUEYNE P022). The intensity was selected based on the threshold at which auricular convulsions were visually observed, and the remaining stimulation conditions were determined based on previous studies (W. He et al., The anticonvulsant effect of transcutaneous auricular vagus nerve stimulation is associated with balancing the autonomic dysfunction in rats, World J. Traditional Chin. Med., vol. 2, no. 3, pp. 48-53, 2016).

The process of extracting HRV parameters from ECG data is described below.

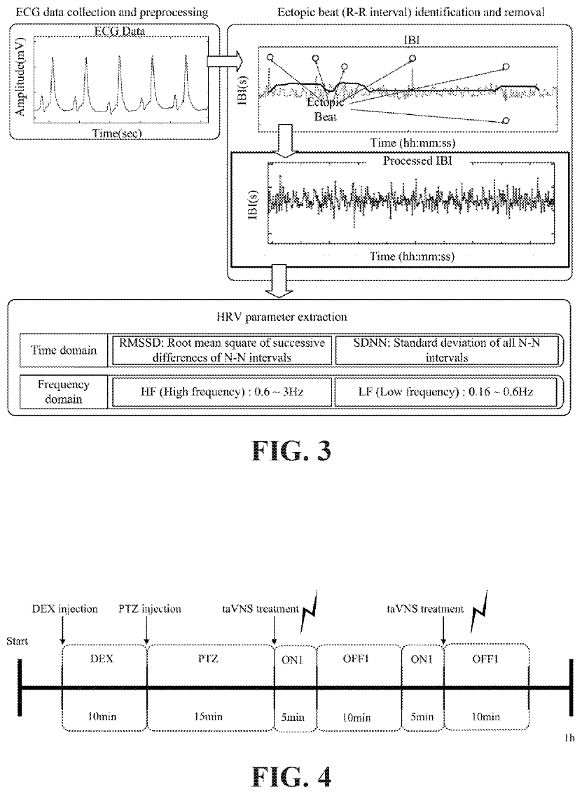

FIG. 3 illustrates an example of a process of extracting HRV parameters from ECG data.

ECG data for all groups were collected for approximately 55 minutes at a sampling rate of 500 Hz. Subsequently, a computing device capable of data processing may be used to extract HRV parameters from the ECG data.

First, the computing device detects R peaks from the ECG data. The computing device applies a 60 Hz notch filter to the ECG data and extracts a tachogram containing R-R intervals required for HRV analysis. The computing device may detect R peaks using the Pan-Tompkins algorithm on the tachogram.

The computing device identifies and removes ectopic R-R intervals from the detected signal. An ectopic beat is a heartbeat that occurs irregularly after a normal heartbeat. An ectopic R-R interval is defined as an interval that deviates by more than 20% from surrounding R-R intervals. As shown in FIG. 3, ectopic R-R intervals correspond to intervals with a large difference in heartbeat intervals compared to surrounding R-R intervals based on the inter-beat interval (IBI). The computing device may remove ectopic R-R intervals from the tachogram.

The computing device may extract HRV parameters in the time domain and frequency domain for the entire interval based on the preprocessed heartbeat interval data.
(1) Time-domain parameters may include the standard deviation of all N-N intervals (SDNN) and the root mean square of successive differences of N-N intervals (RMSSD). Here, N-N intervals correspond to R-R intervals in normal beat signals.
(2) Frequency-domain parameters may include total power (0.16 to 3 Hz), low-frequency power (0.16 to 0.6 Hz), and high-frequency power (0.6 to 3 Hz).

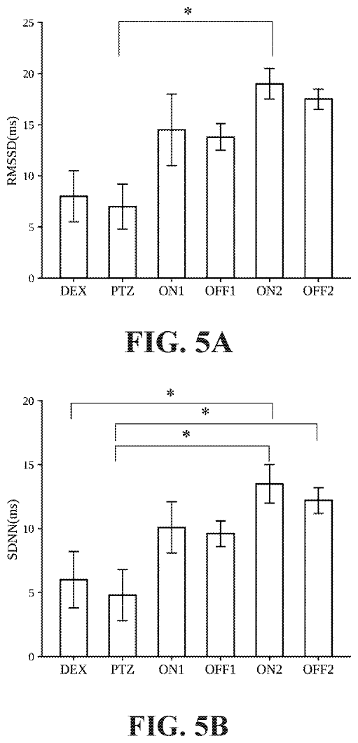

ECG data were collected for 55 minutes as described above. FIG. 4 illustrates an example of intervals in which ECG data were collected. FIG. 4 shows ECG data collection intervals in the taVNS group. In FIG. 4, DEX refers to a 10-minute interval after DEX injection, PTZ refers to a 15-minute interval after PTZ injection, ON1 refers to a 5-minute interval after the first taVNS application (stimulation interval), OFF1 refers to a 10-minute rest interval after the first taVNS ends, ON2 refers to a 5-minute interval after the second taVNS application (stimulation interval), and OFF2 refers to a 10-minute rest interval after the second taVNS ends.

The normal group collected ECG data from the same time intervals as shown in FIG. 4 after DEX injection. The PTZ group collected ECG data from the same time intervals as shown in FIG. 4 after DEX and PTZ injection. That is, the normal group and the PTZ group also collected ECG data from the same time intervals as the taVNS group.

Table 1 below shows the results of extracting HRV parameters from the normal group, PTZ group, and taVNS group.

TABLE 1

| | | HRV | | | | | | F | p |
|---|---|---|---|---|---|---|---|---|---|
| Group | Index | DEX | PTZ | ON1 | OFF1 | ON2 | OFF2 | value | value |
| Normal | RMSSD | 5.300 ± 1.713 | 9.980 ± 2.108 | 9.500 ± 2.017 | 10.980 ± 1.648 | 10.350 ± 1.527 | 9.483 ± 1.038 | 1.412 | 0.255 |
| group | SDNN | 3.980 ± 1.196 | 6.940 ± 1.500 | 6.340 ± 1.217 | 7.400 ± 1.152 | 7.225 ± 1.243 | 6.300 ± 0.528 | 1.177 | 0.349 |
| | HF power | 5.154 ± 2.287 | 14.954 ± 4.547 | 14.220 ± 4.983 | 16.338 ± 4.330 | 15.188 ± 5.187 | 11.868 ± 2.186 | 1.076 | 0.398 |
| | LF power | 11.624 ± 1.961 | 18.764 ± 5.794 | 19.396 ± 6.113 | 15.890 ± 5.399 | 16.428 ± 7.058 | 8.635 ± 3.537 | 0.613 | 0.691 |
| | Total | 5.154 ± 2.287 | 14.954 ± 4.547 | 14.220 ± 4.983 | 16.338 ± 4.330 | 15.188 ± 5.187 | 11.868 ± 2.186 | 0.754 | 0.592 |
| | power | | | | | | | | |
| PTZ | RMSSD | 5.340 ± 0.842 | 8.680 ± 2.759 | 7.780 ± 3.391 | 11.500 ± 3.259 | 10.300 ± 3.003 | 9.167 ± 3.283 | 0.514 | 0.763 |
| group | SDNN | 4.420 ± 0.561 | 6.380 ± 1.967 | 5.620 ± 2.482 | 8.420 ± 2.376 | 7.575 ± 2.170 | 6.950 ± 2.427 | 0.432 | 0.822 |
| | HF power | 3.728 ± 0.936 | 13.866 ± 9.129 | 13.912 ± 9.154 | 25.954 ± 10.030 | 20.493 ± 7.956 | 19.182 ± 10.500 | 0.729 | 0.609 |
| | LF power | 1.148 ± 0.282 | 4.932 ± 3.537 | 6.670 ± 5.784 | 7.088 ± 3.325 | 4.668 ± 2.064 | 6.685 ± 3.745 | 0.373 | 0.862 |
| | Total | 4.876 ± 1.201 | 18.798 ± 12.647 | 20.582 ± 14.792 | 33.042 ± 12.967 | 25.160 ± 8.723 | 25.867 ± 14.242 | 0.603 | 0.698 |
| | power | | | | | | | | |
| taVNS | RMSSD | 8.217 ± 2.782 | 7.067 ± 2.680 | 14.683 ± 3.153 | 14.017 ± 2.075 | 19.067 ± 2.118 | 17.550 ± 1.586 | 3.926 | 0.007 |
| group | SDNN | 6.000 ± 1.837 | 4.850 ± 1.776 | 10.200 ± 2.121 | 9.733 ± 1.332 | 13.500 ± 1.441 | 12.350 ± 0.899 | 4.411 | 0.004 |
| | HF power | 11.492 ± 4.992 | 8.547 ± 4.329 | 31.108 ± 7.959 | 30.132 ± 6.465 | 47.638 ± 8.177 | 39.365 ± 8.976 | 4.780 | 0.002 |
| | LF power | 3.177 ± 1.778 | 2.135 ± 1.272 | 8.885 ± 3.242 | 6.932 ± 2.500 | 14.517 ± 5.109 | 11.872 ± 3.483 | 2.349 | 0.065 |
| | Total | 14.947 ± 6.634 | 12.167 ± 6.825 | 40.002 ± 10.803 | 38.882 ± 9.225 | 74.323 ± 19.639 | 53.258 ± 8.375 | 4.477 | 0.004 |
| | power | | | | | | | | |

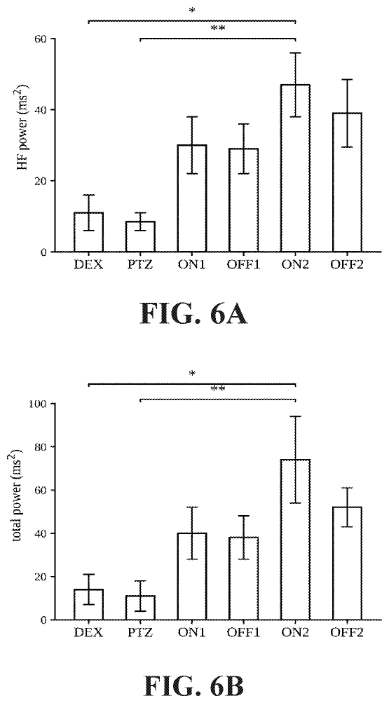

FIGS. 5A and 5B show results of analyzing time-domain HRV variability in the taVNS group. FIG. 5A shows RMSSD (ms) results for the taVNS group, and FIG. 5B shows SDNN (ms) results for the taVNS group.

In the taVNS group, RMSSD and SDNN values increased in the stimulation intervals (including ON1 and ON2) compared to the PTZ administration interval, indicating an increase in parasympathetic activity. In the one-way ANOVA results for the taVNS group, RMSSD showed a significant difference between the PTZ interval (7.067±2.680) and the ON2 interval (19.067±2.118) (p<0.05). SDNN also showed a significant increase between the PTZ interval (4.850±1.776) and the ON2 interval (13.500±1.441) (p<0.05). The numerical values in parentheses represent “mean±standard error.”

In contrast, the normal group and the PTZ group showed no statistically significant differences. Therefore, the results observed in the taVNS group are interpreted as attributable to the taVNS effect independent of factors such as DEX or PTZ.

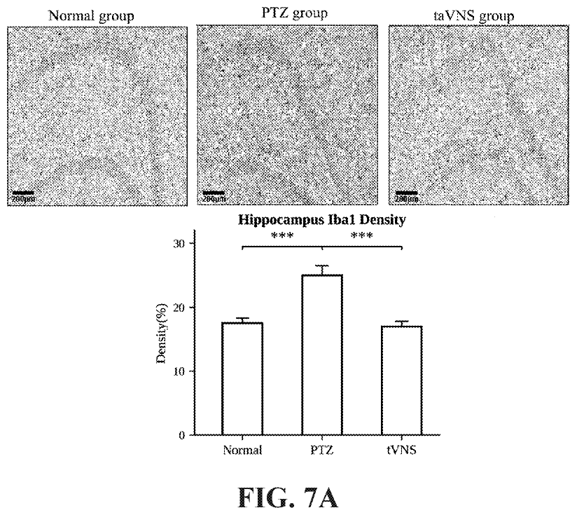

FIGS. 6A and 6B show results of analyzing frequency-domain HRV variability in the taVNS group. FIG. 6A shows high-frequency power (HF power, ms²) results for the taVNS group, and FIG. 6B shows total power (ms²) results for the taVNS group.

In the taVNS group, high-frequency power and total power values increased in the stimulation intervals (including ON1 and ON2) compared to the PTZ administration interval, indicating an increase in parasympathetic activity. In the one-way ANOVA results for the taVNS group, high-frequency power showed a very significant difference between the PTZ interval (8.547±4.329) and the ON2 interval (47.638±8.177) (p<0.01). High-frequency power also showed a significant difference between the DEX interval (11.492±4.992) and the ON2 interval (47.638±8.177) (p<0.05).

In addition, total power showed a very significant difference between the PTZ interval (12.167±6.825) and the ON2 interval (74.323±19.639) (p<0.01). Total power also showed a significant difference between the DEX interval (14.947±6.634) and the ON2 interval (74.323±19.639) (p<0.05).

In contrast, the normal group and the PTZ group showed no statistically significant differences. Therefore, the results observed in the taVNS group are interpreted as attributable to the taVNS effect independent of factors such as DEX or PTZ.

Histological examination was also performed on all rats used in the experiment. Brain tissue was fixed and sectioned from all rats to prepare paraffin sections. Brain tissue was sectioned at a thickness of 4 μm in the coronal plane and Was obtained from regions including the CA1, CA2, CA3, and dentate gyrus of the hippocampus. Iba1, c-Fos, and GAD65 antibodies were selected as markers for neural and synaptic activity. Tissue was visualized with diaminobenzidine (DAB) and counterstained with hematoxylin. The stained brain tissue was observed using Aperio Scan Scope AT, and the number of stained positive cells was quantified. Cell density is defined as the relative ratio (%) of immunopositive cells to the total number of cells.

Changes in the expression of marker proteins were analyzed in the hippocampal region, and expression levels were quantified as the ratio of the number of positive cells to the total number of cells. Table 2 below shows immunohistochemistry data for the normal group, PTZ group, and taVNS group.

TABLE 2

| | Density | | | | |
|---|---|---|---|---|---|
| Protein | Normal group | PTZ group | taVNS group | F value | p value |
| Iba1 | 16.731 ± 0.794 | 25.200 ± 1.844 | 16.708 ± 0.593 | 17.460 | <0.0001 |
| c-Fos | 4.577 ± 1.750 | 12.633 ± 2.808 | 1.363 ± 0.117 | 10.499 | <0.0001 |
| GAD65 | 62.017 ± 2.086 | 60.481 ± 2.549 | 62.809 ± 2.493 | 0.243 | 0.785 |

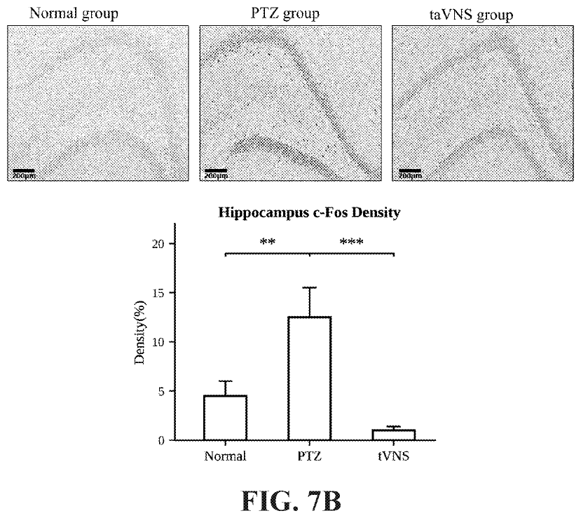

FIGS. 7A and 7B show immunohistochemistry results and analysis of variance results for the normal group, PTZ group, and taVNS group. FIG. 7A shows expression results and analysis of variance results for staining marker Iba1. FIG. 7B shows expression results and analysis of variance results for staining marker c-Fos.

In the one-way ANOVA results in the hippocampal region, Iba-1 density was significantly higher in the PTZ group (25.200±1.844) than in the normal group (16.731±0.794) (p<0.001). In contrast, the taVNS group (16.708±0.593) was significantly lower than the PTZ group (p<0.001). In addition, c-Fos density was significantly higher in the PTZ group (12.633±2.808) than in the normal group (4.577±1.750) (p<0.01), and the taVNS group (1.363±0.117) was significantly lower than the PTZ group (p<0.001).

These results suggest that PTZ induces abnormal neurogenesis during epileptic seizures, and that taVNS, even if it does not completely suppress the epileptic seizures themselves, may protect against cell damage caused by epilepsy.

These experimental results suggest that taVNS is effective in suppressing epileptic seizures, and that the HRV parameters of the subject may serve as an indicator for taVNS therapy.

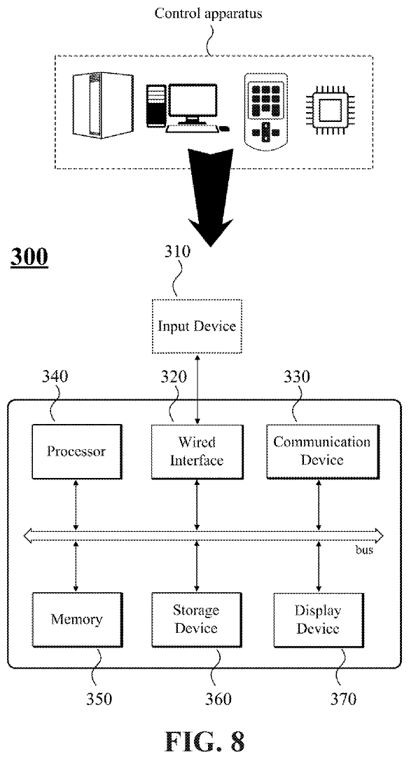

FIG. 8 illustrates an example of a control apparatus 300 for controlling taVNS conditions. The control apparatus 300 may be physically implemented in various forms. (1) The control apparatus 300 may be a device connected to the taVNS device by wire or wirelessly. For example, the control apparatus 300 may take the form of a computer device such as a PC, a smart device, or a network server. (2) The control apparatus 300 may be a device embedded in the taVNS device. For example, the control apparatus 300 may take the form of a chipset with embedded software, a taVNS device, or the like.

The control apparatus 300 may include all or some of the components of FIG. 8 depending on the implementation form. For example, the control apparatus 300 may include a wired interface 320, a processor 340, a memory 350, and a storage device 360. Alternatively, the control apparatus 300 may include a wired interface 320, a communication device 330, a processor 340, a memory 350, and a storage device 360. Alternatively, the control apparatus 300 may include an input device 310, a wired interface 320, a communication device 330, a processor 340, a memory 350, and a storage device 360. Alternatively, the control apparatus 300 may include an input device 310, a wired interface 320, a communication device 330, a processor 340, a memory 350, a storage device 360, and a display device 370.

The input device 310 is a device that receives user commands or information.

The input device 310 may be a device that receives necessary data from an externally connected device or storage device.

The input device 310 may receive ECG data of the epilepsy patient. The ECG data may include ECG data of the epilepsy patient in a normal state and ECG data at the current point in time.

The input device 310 may receive HRV parameters of the epilepsy patient.

The input device 310 may be any one of various types of devices. For example, the input device 310 may be at least one of a mouse, a keyboard, a touch input device, or an external storage medium.

The wired interface 320 is a component that transfers data transmitted by the input device 310 to the inside of the device. The wired interface 320 may consist of a software driver and hardware.

The wired interface 320 may include a controller corresponding to each input device, a device driver that controls the operation of the controller, and a kernel I/O subsystem that integrally manages I/O control requests of the device driver. The kernel I/O subsystem stores I/O requests from device drivers in a queue and schedules the requests based on request priority or device status.

The wired interface 320 may include interfaces such as PS/2, USB (Universal Serial Bus), Ethernet port, HDMI, DisplayPort, and Thunderbolt.

The wired interface 320 may receive ECG data of the epilepsy patient and transfer it to other components of the control apparatus 300.

The wired interface 320 may receive ECG data of the epilepsy patient in a normal state.

The wired interface 320 may receive ECG data of the epilepsy patient at the current point in time (t).

The wired interface 320 may also transfer customized taVNS stimulation parameter information for the target patient to the taVNS device.

The communication device 330 refers to a component that receives and transmits certain information through an external wired or wireless network. The communication device 330 may consist of a circuit including an antenna and a communication module (S/W module, chip, etc.) corresponding to the communication protocol. The communication protocol may be at least one of wired LAN (Ethernet), wireless LAN (IEEE 802.11), mobile communication (LTE, 5G NR, etc.), Bluetooth, and NFC.

The communication device 330 may receive ECG data of the target patient from an external entity.

The communication device 330 may receive ECG data of the epilepsy patient in a normal state.

The communication device 330 may receive ECG data of the epilepsy patient at the current point in time (t).

The communication device 330 may receive HRV parameters of the target patient from an external entity.

The communication device 330 may also transmit customized taVNS stimulation parameter information for the target patient to the taVNS device.

The processor 340 controls the overall operation of the components of the control apparatus 300.

The processor 340 may perform operations on at least one application or computer program for executing methods/operations according to various embodiments of the present disclosure.

The processor 340 is a general-purpose processor that executes at least a portion of a control program installed in the storage device 360 or at least a portion of a program loaded in the memory 350.

The processor 340 may include one or more processors. For example, the processor 340 may include a combination of one or more processors such as a central processing unit (CPU), a microprocessor unit (MPU), a micro controller unit (MCU), a graphic processing unit (GPU), a neural processing unit (NPU), a digital signal processor (DSP), an application processor (AP), or any form of processor well known in the technical field of the present disclosure.

The processor 340 may extract HRV parameters from the ECG data of the epilepsy patient. As in the process described with reference to FIG. 3, the processor 340 may preprocess the ECG data in a certain manner to extract HRV parameters. HRV parameters may include time-domain parameters and frequency-domain parameters. (1) Time-domain parameters may include at least one of the standard deviation of N-N intervals (SDNN) and the root mean square of successive differences of N-N intervals (RMSSD). (2) Frequency-domain parameters may include at least one of total power, low-frequency power, and high-frequency power.

The memory 350 may store data and information generated during the process of the control apparatus 300 controlling taVNS conditions. The memory 350 is a volatile memory such as DRAM or SRAM.

The storage device 360 may store ECG data of the epilepsy patient. The ECG data may include ECG data of the epilepsy patient in a normal state and ECG data at the current point in time.

The storage device 360 may store HRV parameters of the epilepsy patient.

The storage device 360 may store a program that determines taVNS conditions (stimulation intensity, frequency, duration, etc.) based on HRV parameters.

The storage device 360 may store taVNS stimulation parameter information for the epilepsy patient.

The storage device 360 may be implemented as a device such as a hard disk drive, solid state drive, USB flash drive, memory card, optical disc, or network-based storage device (network attached storage, cloud storage, etc.).

The display device 370 is a device that outputs certain information.

The display device 370 may output an interface necessary for the electrical stimulation control process, ECG data, HRV parameters, taVNS intensity, and the like.

The display device 370 may be implemented using various display methods such as liquid crystal, plasma, light-emitting diode (LED), organic light-emitting diode (OLED), surface-conduction electron-emitter, carbon nanotube, and nanocrystal.

The processor 340 may extract HRV parameters from the ECG data of the epilepsy patient. As in the process described with reference to FIG. 3, the processor 340 may preprocess the ECG data in a certain manner to extract HRV parameters. HRV parameters may include time-domain parameters and frequency-domain parameters. (1) Time-domain parameters may include at least one of the standard deviation of N-N intervals (SDNN) and the root mean square of successive differences of N-N intervals (RMSSD). (2) Frequency-domain parameters may include at least one of total power, low-frequency power, and high-frequency power.

Immediately before and during epileptic seizures, HRV parameters show a rapid decrease. The control apparatus may determine electrical stimulation conditions to return the HRV parameters of the epilepsy patient to a normal state.

Equation 1 below is an example of a formula for modeling HRV parameter changes.

E(t)=HRV_{Baseline}−HRV_{seizure}(t) [Equation 1]

E(t) represents HRV or the change in HRV parameters. HRV_{baseline} is the HRV parameter of the epilepsy patient in a normal state. HRV_{seizure} is the HRV parameter of the epilepsy patient during a seizure. t refers to a specific point in time. Therefore, E(t) represents the amount of change in HRV.

Equation 2 below is an example of a formula for modeling taVNS electrical stimulation parameters. Equation 2 is based on a PID control structure. PID control outputs control information for the object to be controlled based on a certain input value.

$$Stim ( t ) = {K}_{p} E ( t ) - ( {K}_{i} \int E ( t ) dt + {K}_{d} \frac{dE ( t )}{dt} ) \qquad [ Equation 2 ]$$

Stim(t) is stimulation parameter information for taVNS control at time t. The stimulation parameter information may include at least one of stimulation intensity, frequency, and duration. Kₚ is the proportional gain, which adjusts stimulation in proportion to the magnitude of the HRV error. Kᵢ is the integral gain, which is for correction against long-term accumulation of HRV error. K_{d} is the derivative gain, which is for correction against the rate of change of HRV error (rapid change).

The PID (Proportional-Integral-Derivative) controller of Equation 2 seeks to minimize the HRV parameter error E(t) by applying stimulation Stim(t) at time t. Stimulation Stim(t) corresponds to an electrical stimulation condition for returning the HRV parameter to a normal state.

The PID controller of Equation 2 does not merely implement an abstract mathematical formula. Rather, each gain parameter is calibrated to address specific physiological phenomena observed in epileptic patients: (i) the proportional gain Kₚ provides immediate corrective stimulation proportional to the current HRV deviation, counteracting the acute reduction in parasympathetic tone that accompanies seizure onset; (ii) the integral gain Kᵢ accumulates and corrects sustained HRV deficits over time, addressing the chronic autonomic dysfunction present in epileptic patients even between seizures; and (iii) the derivative gain Kₑ responds to the rate of change of HRV deviation, enabling anticipatory stimulation adjustment in response to rapidly evolving seizure activity.

As a result, the PID-based control of taVNS conditions directly causes physical changes in the electrical stimulation delivered to the auricular branch of the vagus nerve (ABVN), which in turn activates vagal afferent fibers projecting to the nucleus tractus solitarius (NTS), thereby producing measurable improvements in autonomic function and providing neuroprotective effects in the hippocampus. This causal chain from computed stimulation parameters to physical electrical stimulation to physiological outcome constitutes an integration of the mathematical relationship into a practical application in the medical device field.

The processor 340 may compute the change amount of HRV parameters at the current point in time t according to Equation 1.

The processor 340 may compute taVNS stimulation parameter information that minimizes the HRV parameter error E(t) according to the PID control of Equation 2.

The processor 340 may determine stimulation conditions by referring to a pre-defined stimulation parameter map based on the change amount of HRV parameters (E(t)). The stimulation parameter map includes stimulation intensity, frequency, and stimulation duration corresponding to each section of the HRV change amount, through which the control apparatus may perform continuous and nonlinear stimulation control according to changes in the patient's condition.

The methods according to the embodiments described in the specification of the present disclosure may be implemented in the form of hardware, software, or a combination of hardware and software.

When implemented in software, a computer-readable storage medium storing one or more programs (software modules) may be provided. One or more programs stored in the computer-readable storage medium are configured for execution by one or more processors in an electronic device. The one or more programs include instructions for causing the electronic device to execute methods according to the embodiments described in the specification of the present disclosure.

In addition, the HRV parameter-based taVNS intensity determination method or taVNS control method described above may be implemented as a program (or application) including an executable algorithm that can be executed by a computer. The program may be stored and provided in a transitory or non-transitory computer-readable medium.

The non-transitory computer-readable medium refers to a medium that semi-permanently stores data and is readable by a device, as distinguished from a medium such as a register, cache, or memory that stores data for a short period of time. Specifically, the various applications or programs described above may be stored and provided in non-transitory computer-readable media such as a compact disc (CD), a digital versatile disc (DVD), a hard disk, a Blu-ray disc, a universal serial bus (USB), a memory card, a read-only memory (ROM), a programmable ROM (PROM), an erasable PROM (EPROM), an electrically EPROM (EEPROM), or a flash memory.

The transitory computer-readable medium refers to various types of random-access memory (RAM) such as static RAM (SRAM), dynamic RAM (DRAM), synchronous DRAM (SDRAM), double data rate SDRAM (DDR SDRAM), enhanced SDRAM (ESDRAM), Synclink DRAM (SLDRAM), and Direct Rambus RAM (DRRAM).

In addition, the program may be stored in an attachable storage device accessible through a communication network such as the Internet, an intranet, a local area network (LAN), a wide area network (WAN), or a storage area network (SAN), or a combination thereof. Such a storage device may connect to a device implementing the embodiments of the present disclosure through an external port. In addition, a separate storage device on a communication network may connect to a device implementing the embodiments of the present disclosure.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of providing vagus nerve electrical stimulation to an epilepsy patient, the method comprising:
receiving, by a transcutaneous auricular vagus nerve stimulator, electrocardiogram (ECG) data of an epilepsy patient at a specific point in time;
extracting, by the transcutaneous auricular vagus nerve stimulator, a heart rate variability (HRV) parameter from the ECG data;
computing, by the transcutaneous auricular vagus nerve stimulator, a change amount between an HRV parameter in a normal state of the epilepsy patient and an HRV parameter at the specific point in time;
generating, by the transcutaneous auricular vagus nerve stimulator, stimulation parameter information for vagus nerve electrical stimulation of the epilepsy patient based on the change amount; and
providing, by the transcutaneous auricular vagus nerve stimulator, vagus nerve electrical stimulation to the epilepsy patient according to the stimulation parameter information.

2. The method of claim 1, wherein the HRV parameter comprises a time-domain parameter and a frequency-domain parameter,
wherein the time-domain parameter comprises a standard deviation of N-N intervals and a root mean square of successive differences of N-N intervals, and
wherein the frequency-domain parameter comprises at least one of total power, low-frequency power, and high-frequency power.

3. The method of claim 1, wherein the transcutaneous auricular vagus nerve stimulator determines the stimulation parameter information for the vagus nerve electrical stimulation based on the change amount using a proportional-integral-derivative (PID) control technique.

4. The method of claim 1, wherein the transcutaneous auricular vagus nerve stimulator determines the stimulation parameter information for the vagus nerve electrical stimulation using the following equation:
Stim(t)=KₚE(t)−(Kᵢ∫E(t)dt+K_{d}dE(t)/dt)
where t is the specific point in time, Stim(t) is the stimulation parameter information for the vagus nerve electrical stimulation, Kp is a proportional gain, Ki is an integral gain, Kd is a derivative gain, and E(t) is the change amount.

5. The method of claim 1, wherein the stimulation parameter information for the vagus nerve electrical stimulation comprises at least one of stimulation intensity, frequency, pulse width, and stimulation duration.

6. The method of claim 1, wherein the stimulation parameter information for the vagus nerve electrical stimulation is set to increase stimulation intensity as the HRV parameter decreases.

7. The method of claim 1, wherein the transcutaneous auricular vagus nerve stimulator updates the stimulation parameter information for the vagus nerve electrical stimulation using HRV parameters extracted from consecutive time intervals.

8. A method of controlling vagus nerve electrical stimulation for an epilepsy patient, the method comprising:
receiving, by a control apparatus, electrocardiogram (ECG) data of an epilepsy patient at a specific point in time;
extracting, by the control apparatus, a heart rate variability (HRV) parameter from the ECG data;
computing, by the control apparatus, a change amount between an HRV parameter in a normal state of the epilepsy patient and an HRV parameter at the specific point in time; and
determining, by the control apparatus, stimulation parameter information for vagus nerve electrical stimulation of the epilepsy patient based on the change amount,
wherein the stimulation parameter information for the vagus nerve electrical stimulation comprises at least one of stimulation intensity, frequency, pulse width, and stimulation duration.

9. The method of claim 8, wherein the HRV parameter comprises a time-domain parameter and a frequency-domain parameter,
wherein the time-domain parameter comprises a standard deviation of N-N intervals and a root mean square of successive differences of N-N intervals, and
wherein the frequency-domain parameter comprises at least one of total power, low-frequency power, and high-frequency power.

10. The method of claim 8, wherein the control apparatus determines the stimulation parameter information for the vagus nerve electrical stimulation based on the change amount using a proportional-integral-derivative (PID) control technique.

11. A control apparatus for vagus nerve electrical stimulation for an epilepsy patient, the control apparatus comprising:
an interface device configured to receive electrocardiogram (ECG) data of an epilepsy patient at a specific point in time;
a storage device configured to store ECG data of the epilepsy patient in a normal state; and
a processor configured to compute a change amount between a heart rate variability (HRV) parameter in the normal state derived from the ECG data in the normal state of the epilepsy patient and an HRV parameter at the specific point in time derived from the received ECG data, and to compute stimulation parameter information for vagus nerve electrical stimulation of the epilepsy patient based on the change amount.

12. The control apparatus of claim 11, wherein the HRV parameter comprises a time-domain parameter and a frequency-domain parameter,
wherein the time-domain parameter comprises a standard deviation of N-N intervals and a root mean square of successive differences of N-N intervals, and
wherein the frequency-domain parameter comprises at least one of total power, low-frequency power, and high-frequency power.

13. The control apparatus of claim 11, wherein the processor determines the stimulation parameter information for the vagus nerve electrical stimulation based on the change amount using a PID control technique.

14. The control apparatus of claim 11, wherein the processor computes the stimulation parameter information for the vagus nerve electrical stimulation using the following equation:
$$Stim ( t ) = {K}_{p} E ( t ) - ( {K}_{i} \int E ( t ) dt + {K}_{d} \frac{dE ( t )}{dt} )$$
where t is the specific point in time, Stim(t) is the stimulation parameter information for the vagus nerve electrical stimulation, Kp is a proportional gain, Ki is an integral gain, Kd is a derivative gain, and E(t) is the change amount.

15. The control apparatus of claim 11, wherein the stimulation parameter information for the vagus nerve electrical stimulation comprises at least one of stimulation intensity, frequency, pulse width, and stimulation duration.
